(19) United States
(12) Patent Application Publication
(10) Pub. No.: US 2006/0141620 A1
(43) Pub. Date: Jun. 29, 2006

(54) System and method for forming a cardiac tissue construct

(75) Inventors: David L. Brown, Ann Arbor, MI (US); Ravi K. Birla, Ann Arbor, MI (US); Robert G. Dennis, Chapel Hill, NC (US); Gregory H. Borschel, Toronto (CA)

Correspondence Address: BROOKS KUSHMAN P.C., 1000 TOWN CENTER, TWENTY-SECOND FLOOR, SOUTHFIELD, MI, 48075, US

(73) Assignee: The Regents of the University of Michigan, Ann Arbor, MI (US)

(21) Appl. No.: 11/274,667

(22) Filed: Nov. 15, 2005

Related U.S. Application Data
(60) Provisional application No. 60/627,941, filed on Nov. 15, 2004.

(51) Int. Cl. C12N 5/08; C12M 1/00

(52) U.S. Cl. 435/366; 435/289.1

(57) ABSTRACT

A system and method for forming a cardiac tissue construct includes a chamber and cardiac myocytes provided within the chamber. The chamber is arranged to at least partially surround an intact blood vessel in vivo to facilitate formation of the three-dimensional cardiac tissue construct within the chamber.

### CROSS-REFERENCE TO RELATED APPLICATIONS

This application claims the benefit of U.S. provisional application Ser. No. 60/627,941 filed Nov. 15, 2004.

### STATEMENT REGARDING FEDERALLY SPONSORED RESEARCH OR DEVELOPMENT

The invention was made with Government support under Contract No. N66001-02-C-8034 from DARPA. The Government has certain rights to the invention.

### BACKGROUND OF THE INVENTION

1. Field of the Invention

This invention relates to a system and method for forming a cardiac tissue construct.

2. Background Art

Congestive heart failure (CHF) is a major medical challenge, with 4.8 million patients in the U.S. currently suffering from its effects. Current therapeutic strategies used to treat CHF include pharmaceutical intervention, mechanical cardiac support, and heart transplantation. Although these treatments have significantly improved the quality of patient care, there is a need for improved options. For example, heart transplantation has been the most successful modality in the treatment of severe CHF, with over 55,000 heart transplantations being performed as of March 2001, and a short term survival rate of 81%. However, widespread applicability is limited by the chronic shortage of donor organs.

Cardiac tissue engineering may offer an alternative treatment modality for CHF and other heart ailments by providing tissues to replace ischemic or scarred myocardium. Engineered myocardial tissue could also be used to patch myocardial septal defects and for reconstruction of congenital cardiac anomalies. In addition to its potential clinical applicability, engineered three-dimensional cardiac tissue could be useful as a system for basic cardiology research and as a means to evaluate efficacy and safety in new drug development.

There have been several strategies utilized to engineer three-dimensional cardiac tissue constructs. Temperature sensitive surfaces have been used to manufacture two-dimensional monolayers of cardiac myocytes, which can then be stacked together to form three-dimensional cardiac muscle constructs (see Shimizu et al., Tissue Eng. 7(2): 141-151, 2001; Shimizu et al., Circ. Res. 90(3): e40-e48, 2002; Shimizu et al., J. Biomed. Mat. Res. 60(1): 110-117, 2002). Synthetic scaffolds fabricated from polyglycolic acid have been used to generate three-dimensional constructs by seeding with neonatal cardiac myocytes (see Bursac et al., Am. J. Physiol. 277(2pt2): H433-H444, 1999; Carter et al., Biotech. & Bioeng. 64(5): 580-589, 1999; Papadaki et al., Am. J. Physiol.—Heart & Circ. Physiol. 280(1): H168-H178, 2001). In addition, others have investigated various biodegradable gel systems consisting of gelatin (see Li et al., Circulation 100(19): Suppl II-63-II-69, 1999: Li et al., J. Thor. & Cardiovasc. Surg. 119(2): 368-375, 2000; Sakai et al., J. Thor. & Cardiovasc. Surg. 121(5): 932-942, 2001) alginate (see Leor et al., Circulation 102(19): Suppl III-56-III-61, 2000) and collagen (see Eschenhagen et al., FASEB J. 11(8): 683-694, 1997; Souren et al., In Vitro Cell. & Dev. Biol. 28A(3part1): 199-204, 1992; Zimmermann et al., Biotech. & Bioengin. 68(1): 106-114, 2000; Zimmermann et al., Circ. Res. 90(2): 223-230, 2002).

An in vitro method for engineering cardiac tissue from neonatal cardiac myocytes has been described previously in U.S. Patent Application Publication No. 2004/0132184. The resulting three-dimensional tissue constructs, termed “cardioids”, can be electrically stimulated to generate significant active force, such as on the order of 75 μN, and can be electrically paced at frequencies of up to approximately 100 Hz without fused tetanus. The cardioids exhibit positive inotropy in response to ionic calcium and positive chronotropy in response to epinephrine.

As tissue engineered cardiac constructs increase in scale, their size may be limited by the ability to supply nourishment to the cells in the center of the construct. The diffusion of oxygen places a major design constraint on all avascular engineered tissues. Angiogenesis becomes necessary to engineer tissue constructs with a diameter greater than 200 μm (see Colton, Cell Transplant. 4(4): 415-436, 1995). Therapeutic angiogenesis is the process by which local growth of blood vessels is induced by various interventions, resulting in neo-vascularization of engineered constructs (see Hockel et al., Arch. Surg. 128(4): 423-429, 1993; Soker et al., World J. Urol. 18(1): 10-18, 2000). There are several strategies that have resulted in initial success at therapeutic angiogenesis in engineered constructs. Incorporation of angiogenic factors (see Epstein et al., Cardiovasc. Res. 49(3): 532-542, 2001; Shea et al., Nature Biotech. 17(6): 551-554, 1999) seeding of endothelial cells (see Holder et al., Tissue Eng. 3(2): 149-160, 1997) pre-vascularization of the scaffold prior to cell seeding (see Mikos et al., Biotech. & Bioeng. 42(6): 716-723, 1993) or the use of solid free-form fabrication (see Griffith et al., Ann. N.Y. Acad. Sci. 831: 382-397, 1997; Hollister et al., Int. J. Oral & Maxillofacial Surg. 29(1): 67-71, 2000) have all been evaluated.

One approach to introducing a vascular supply to three-dimensional engineered tissues has been shown to support survival and function of engineered tissues (see Cronin et al., Plast. Reconstr. Surg. 113(1): 260-269, 2004; Cassell et al., Ann. N.Y. Acad. Sci. 944: 429-442, 2001; Mian et al., Tissue Eng. 6(6): 595-603, 2000; Tanaka et al., Br. J. Plast. Surg. 53(1): 51-57, 2000). Angiogenesis from arteriovenous loop and flow-through vascular pedicle models has been demonstrated to result in the creation of a newly-formed “flap” of tissue. This model has been shown to support pancreatic islet cells and adipocytes (see Mian et al., Tissue Eng. 7(1): 73-80, 2001).

### BRIEF DESCRIPTION OF THE DRAWINGS

FIG. 1 is a photograph of a chamber (C) containing a fibrin gel suspension of cardiac cells implanted around the femoral artery (A) and vein (V) in the groin of a rat, wherein IL is the inguinal ligament;

FIG. 2 is a photograph of a cardiac tissue construct (C) according to the present invention after removal from the chamber, wherein the construct (C) is placed in a bath of culture media with one end pinned stationary to the culture plate (P) and the other end attached to a force transducer (FT), and stimulation is accomplished with parallel platinum electrodes (E);

FIG. 3 is a graph of the active force generated by cardiac tissue constructs according to the present invention, wherein the constructs were stimulated with 5V at a 10 ms pulse width (S) to elicit a twitch contraction (C);

FIG. 4 is a graph depicting electrical pacing of cardiac tissue constructs at 1 Hz according to the present invention;

FIG. 5 is a graph illustrating the length-force relationship of cardiac tissue constructs according to the present invention, wherein the length of each construct was optimized for maximum force production and the force measured at every other length was normalized to the force at the optimal length, and wherein values shown represent the mean and standard deviation of five force readings for a single construct;

FIG. 6 is a graph showing the inotropic response to ionic calcium of cardiac tissue constructs according to the present invention, wherein following complete removal of calcium from the system, calcium was reintroduced incrementally and force production was tested, where values shown represent the mean and standard deviation of five force readings for a single construct;

FIG. 7 is a graph of the chronotropic response of cardiac tissue constructs according to the present invention to epinephrine, wherein the behavior shown is representative of four constructs tested in this manner;

FIGS. 8A-8D are photomicrographs demonstrating histology of cardiac tissue constructs according to the present invention: FIGS. 8A and 8B show H&E staining of a section through the center of a construct which demonstrates large areas of viable muscle tissue (M), multiple vascular channels of all sizes permeating the construct (arrows), and red blood cells, and FIGS. 8C and 8D show von Willebrand staining of constructs which highlights the endothelial lining within the newly formed vascular channels (arrows);

FIGS. 9A and 9B are electron micrographs (EM) of cardiac tissue constructs according to the present invention: FIG. 9A is an EM section of a single cardiac myocyte within the construct showing the presence of well aligned myofilaments (myo), equally spaced z-lines (Z-line), abundant amounts of mitochondria (mito) which are closely associated to the myofilaments, and the presence of collagen (coll) fibers, and FIG. 9B is an EM section of a construct showing the presence of seven cardiac myocytes (CM1-7) with a large number of intercalated discs (arrows) formed between myocytes, wherein nuclei (Nuc) of the cardiac myocytes as well as myofilaments with associated mitochondria are also evident;

FIG. 10 is a schematic representation of a testing apparatus for determining the internal pressure generated by cardiac tissue constructs according to the present invention;

FIG. 11 is a graph of the twitch pressure generated by cardiac tissue constructs according to the present invention;

FIGS. 12A-B are graphs of the starling behavior of the cardiac tissue constructs according to the present invention, wherein the baseline pressure of the constructs was gradually decreased with a resultant decrease in the active twitch pressure; and

FIG. 13 is a graph of the starling curve for cardiac tissue constructs according to the present invention.

### DETAILED DESCRIPTION OF THE INVENTION

As required, detailed embodiments of the present invention are disclosed herein; however, it is to be understood that the disclosed embodiments are merely exemplary of the invention that may be embodied in various and alternative forms. The figures are not necessarily to scale, some features may be exaggerated or minimized to show details of particular components. Therefore, specific structural and functional details disclosed herein are not to be interpreted as limiting, but merely as a representative basis for the claims and/or as a representative basis for teaching one skilled in the art to variously employ the present invention.

The present invention includes a contractile, three-dimensional cardiac tissue construct along with a system and method of engineering the construct via association with an intrinsic vascular supply. By way of example, the cardiac tissue construct and system and method for its production according to the present invention are described with reference to the use of tissue harvested from and subsequently implanted in rats. However, it is fully contemplated that tissue from any mammal, including human beings, could be similarly utilized according to the method described herein. The construct, system and method of the present invention are not intended to be limited to one particular cell origin or age, construct shape, time frame, component concentration, or culture condition. One skilled in the art can readily appreciate that various modifications can be made to the construct, system and method described herein without departing from the scope of the invention disclosed.

Cardiac myocytes may be isolated from heart tissue, such as those from 2-3 day old F344 rats (Charles River Laboratories, Inc., Wilmington, Mass.). According to one aspect of the present invention, heart ventricles may be removed, separated from the atria, and trisected (see Boluyt et al., Circulation Research 81(2): 176-186, 1997). Of course, other methods for obtaining cardiac tissue are also fully contemplated. Unless otherwise specified, materials described below may be purchased from Sigma-Aldrich (St. Louis, Mo.). Cardiac tissue may be cut into pieces (for example, ˜2 mm cubes) and suspended in a dissociation solution (DS) which may include approximately 0.32 mg/ml collagenase type II (Worthington Biochemical Corporation, Lakewood, N.J.) and approximately 0.6 mg/ml pancreatin dissolved in a buffer including ˜116 mM NaCl, ˜20 mM HEPES, ˜1 mM Na₂HPO₄, ˜5.5 mM glucose, ˜5.4 mM KCl and ˜0.8 mM MgSO₄. Digestion may be carried out in an orbital shaker for about 5 minutes at ˜37° C., after which the supernatant may be replaced with fresh DS and the digestion process may be continued for approximately an additional 30 minutes.

At the end of the digestion process, the supernatant may be collected in approximately 5 ml of horse serum (Invitrogen Corporation, Auckland, New Zealand), centrifuged at approximately 1500 rpm for ˜5 minutes, and the cell pellet may be resuspended in approximately 5 ml horse serum. Fresh DS may be added to the original, undigested tissue and the digestion process may be repeated an additional 2-3 times. Cells from all the digests may be pooled, centrifuged and then suspended in plating medium including approximately 335 ml Dulbecco's modification of Eagle's medium (DMEM), ˜85 ml M199, ˜25 ml fetal bovine serum, ˜50 ml horse serum, and ˜5 ml antibiotic-antimycotic (Invitrogen). The total number of cells may be determined using a hemocytometer. Isolated cardiac myocytes may be suspended in fibrin gel (including a ratio of approximately 200 μl of 3.5 mg/ml fibrinogen to approximately 5 μl of 200 U/ml of thrombin) at a concentration of about twenty million cells (which may include cardiac myocytes, cardiac fibroblasts, vascular endothelial cells, and possibly smooth muscle cells) per 50 μl of fibrin gel. Thrombin may promote the formation of the fibrin gel.

The cell suspensions may then be loaded into chambers. Silicone or other tubing (Cole Parmer Instrument Company, Vernon Hills, Ill.) may be utilized having, for example, but not limited to, dimensions of approximately 1.6 mm ID, approximately 3.1 mm OD, and a wall thickness of approximately 0.75 mm may be cut to a desired length, such as 10 mm, which would result in an internal volume of ˜50 μl. Of course, other types of chambers or chambers of smaller or larger volumes, and thus constructs of smaller or larger volumes, are also fully contemplated. There should be no limitations on the length or diameter of the constructs that can be created. An opening, such as a longitudinal slit, may be made within each tube to allow it to be separated for placement around intact vessels in vivo as described below. Approximately 50 μl of the cardiac cell suspension may be pipetted into the lumen of each chamber, which may be vertically-oriented, and allowed to gel. Control constructs may be prepared by filling chambers with fibrin gel without cells.

It is understood that all reagent measurements, materials, submersion times, and other values described above are approximate, and can be reasonably varied without affecting the method and resulting constructs. Furthermore, the approximate volumes of reagents within solutions described above may be altered to provide a solution with reagents having similar volume ratios.

Following preparation of the gel-filled chambers, the chambers may be implanted in a host at least partially around an intact blood vessel, such as by passing an intact vascular pedicle through the slit in the side of the tubes, and incubated for a period of time to allow for formation of the cardiac tissue construct. The association of a vascular pedicle within the forming construct may induce angiogenesis, resulting in more thorough vascularization of the tissues. The use of the chamber may keep the introduced cells in relative isolation and confine the final construct to a more useable and testable form. The chambers also hold the cells in close proximity to one another, and may allow them to organize and form intracellular connections. Spontaneous matrix deposition within the chambers may provide enhanced mechanical integrity to the cardiac tissue constructs.

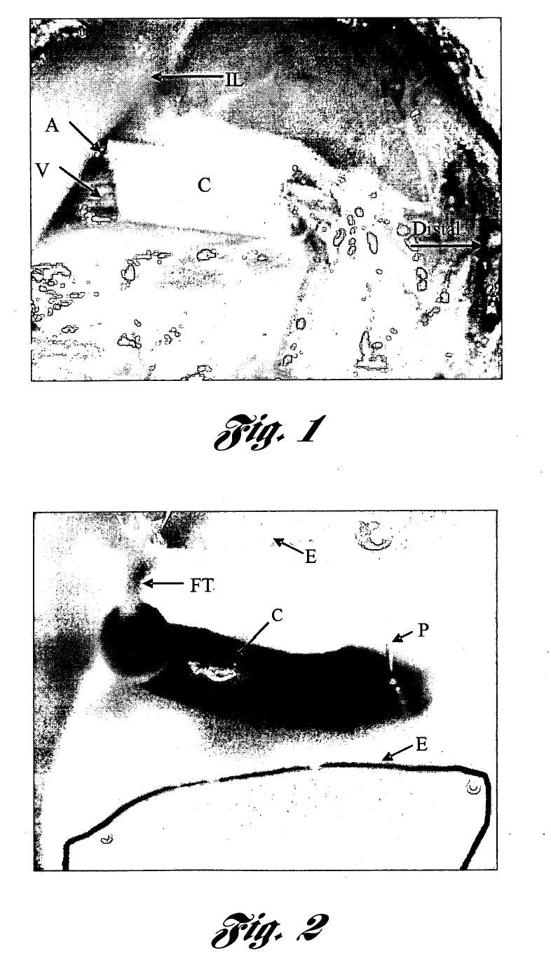

In testing the method and resulting constructs formed according to the system and method of the present invention, 6 cardiac myocyte-seeded chambers and 6 control chambers were implanted in vivo into recipient adult F344 rats weighing approximately 230-270 grams. The animals were anesthetized and the groins were clipped of hair and sterilely prepped and draped. The femoral vessels were exposed through a longitudinal incision made on the medial thigh. With the aid of a dissecting microscope, the femoral artery and vein may be separated from their surrounding tissue attachments and the accompanying nerve. Several small vascular branches to the thigh musculature were cauterized to isolate the length of the vessels from the inguinal ligament to the branch point of the epigastric vessels. The cardiac myocyte/fibrin gel-filled chambers and control chambers were placed around the femoral vessels (FIG. 1). The wounds were closed in two layers over the chambers and the animals were allowed to recover.

After three weeks of in vivo implantation, the rats were re-anesthetized and the chambers were surgically exposed. Of course, implantation duration could be varied in accordance with the present invention. At 3 weeks, all of the cell-filled chambers were found to be completely filled with vascularized living cardiac tissue, where the femoral vessels could be seen within the substance of the tissue. Patency of the vascular pedicles was confirmed by visually monitoring the blood flow through the vessels both before and after pedicle division. Removal of the resulting three-dimensional cardiac constructs according to the present invention from the chambers may be accomplished by dividing the femoral artery, femoral vein, and all soft tissue attachments at the proximal and distal ends of the chambers, and then opening the chambers along their longitudinal slit. Of course, alternative methods of removal are also contemplated. Following removal, the cardiac tissue constructs were pinned to 35 mm tissue culture plates coated with polydimethylsiloxane substrate (Dow Chemical Corporation, Midland, Mich.). Culture medium (CM) including approximately 365 ml DMEM, ˜100 ml M199, ˜35 ml fetal bovine serum, and ˜5 ml antibiotic-antimycotic (Invitrogen) was added to the plates.

Upon explantation, each of the experimental cardiac muscle constructs exhibited spontaneous contractility. When removed from the chambers and placed in the CM-filled culture dishes at 37° C., the cardiac muscle constructs flexed back and forth along their long axis at approximately 0.5 Hz. This spontaneous contractile activity lasted for approximately five minutes, ultimately slowing to a stop.

The six control chambers, manufactured without the addition of cardiac cells, were also harvested after three weeks of in vivo implantation around the femoral vessels. The femoral vessels remained patent in all cases. No spontaneous contraction occurred upon explantation and there was no active force generation upon electrical stimulation.

In order to evaluate the contractility of the cardiac tissue constructs, electrical stimulation was applied to the constructs by placing them between parallel platinum wire electrodes in the CM-filled plates at 37° C. (FIG. 2). One end of the construct was fixed to the plate and the other end was attached to a custom-built optical force transducer. Stimulated twitch force measurements were then recorded at 5 volts and a 10 ms pulse width. The length of each construct was adjusted to obtain maximum active force using a multi-axis micromanipulator. This optimal length was designated as Lₒ and was recorded. Construct lengths and diameters were measured with a calibrated eyepiece reticle with a resolution of 5 μm. The cross-sectional area (CSA) of each construct was calculated on the basis of the measured diameter, with the assumption that the construct approximated a cylinder.

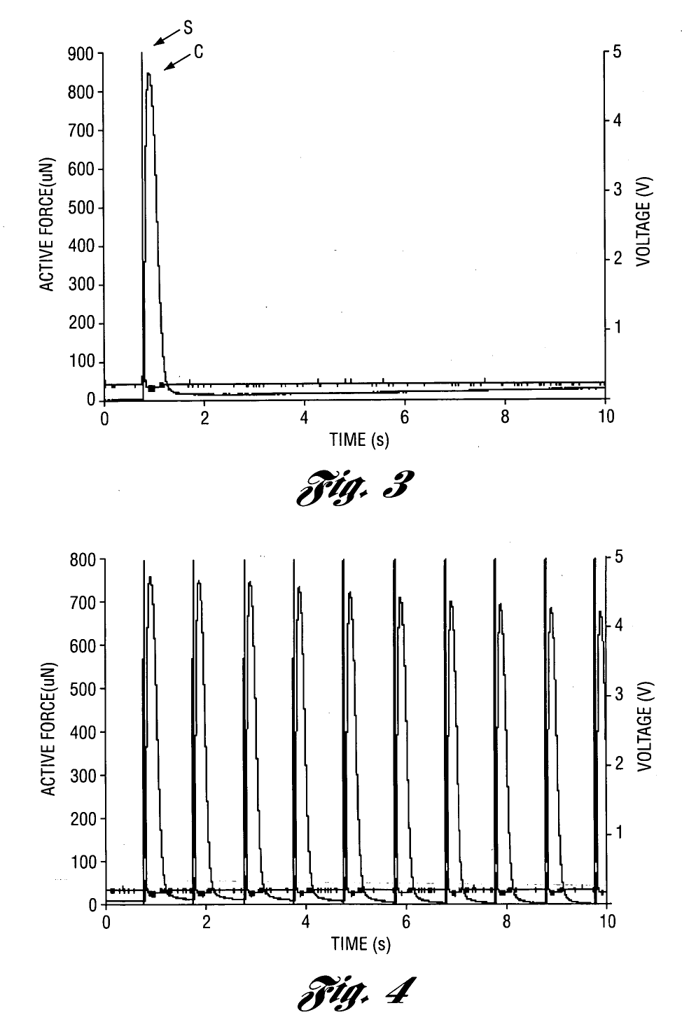

As depicted in FIG. 3, the cardiac tissue constructs according to the present invention are electrically excitable, where the average twitch force of the constructs was found to be 263 μN (n=5), with a maximum force recorded for one construct of 843 μN. The average specific force, determined by normalizing the peak active force by CSA, was found to be 2.03 kPa (n=5) with a maximum value of 6.45 kPa.

To characterize the cardiac-like contractility characteristics of the cardiac muscle constructs according to the present invention, the constructs were subjected to electrical pacing at frequencies from 1 to 20 Hz, with all other stimulation parameters remaining constant (FIG. 4). The cardiac constructs were capable of generating repetitive contractions, as active force was generated following each electrical stimulus delivered. During the course of electrical pacing, a slight decrease in the peak active force was observed with each subsequent active contraction. This is indicative of muscle fatigue and may be due to nutrient deprivation during evaluation in vitro. However, the cardiac constructs completely relaxed between contractions, similar to normal cardiac muscle, with active forces returning back to baseline resting tension between each contraction.

The equilibrium length-force relationship was evaluated by gradually distracting the distance between the two ends of the cardiac constructs as loaded on the force transducer, beginning at the resting length of the construct. Stimulated force measurements were obtained at incremental specimen lengths and were compared to the optimal length Lₒ. As depicted in FIG. 5, increasing the length of the constructs initially resulted in a proportional increase in force generation. This increase eventually reached a plateau, and then decreased proportionately with further incremental lengthening. Accordingly, the constructs according to the present invention exhibited a length-force relationship characteristic of muscle tissue, with force tracings showing an ascending limb, a plateau region and a descending limb with increasing length.

The effect of the addition of exogenous calcium on contractility was investigated in the constructs according to the present invention. First, CM in the bath was replaced with calcium-free DMEM (Invitrogen). Known volumes of 100 mM ethylene glycol-bis(2-aminoethylether)-N,N,N′,N′-tetraacetic acid (EGTA) were then added to the bath to bind residual stores of calcium in the constructs. At each incremental addition of EGTA, twitch stimulation was applied and the force of contraction was measured. Removal of calcium was considered to be complete when the force of contraction was not detectable and no further EGTA was added. At this point, the calcium-free DMEM in the bath was aspirated and the construct was washed 3 times with fresh calcium-free DMEM. Known volumes of 100 mM calcium were then added and stimulated contraction forces were re-evaluated with each addition. This process was repeated until there was no further increase in the active force with the addition of calcium. The relationship of calcium concentration of the bath to force of contraction was then plotted.

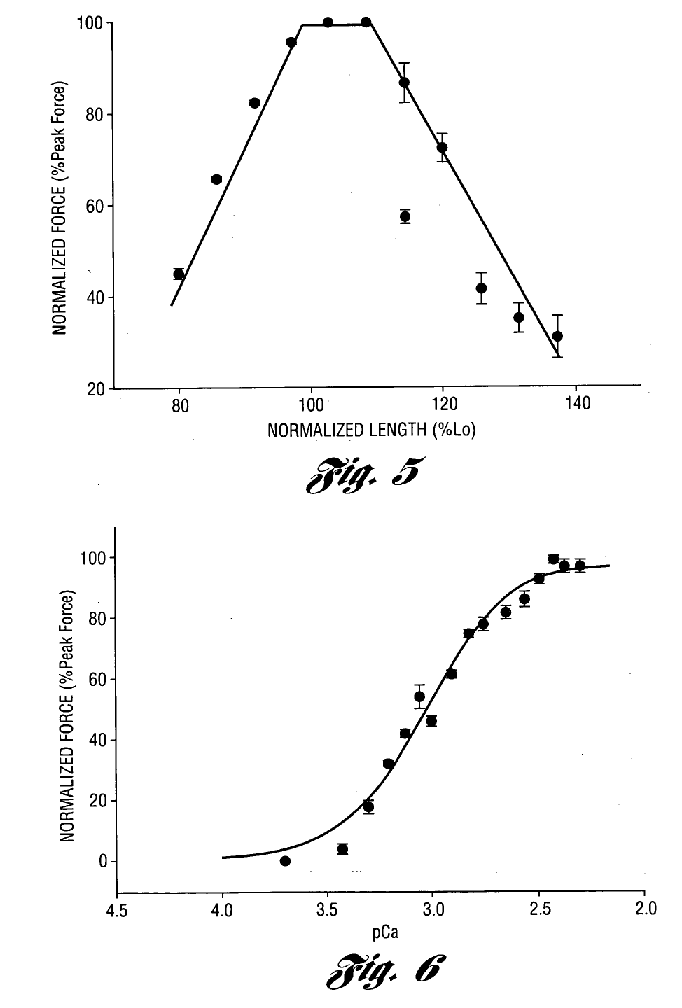

As shown in FIG. 6, the active force was not significantly different than the noise level at very low calcium concentrations in the bath. As the calcium concentration was increased, incremental additions of ionic calcium resulted in proportional rises in active force production. A plateau was reached, at which point further addition of calcium did not produce an increase in active force production. Accordingly, explanted constructs exhibited a sigmoidal responsiveness to external calcium, where the pCa_{1/2} of the constructs was found to be 3.04. The pCa_{1/2} of mammalian cardiac tissue has been reported in the range 5.9 to 7.0 (see Bers, Excitation-Contraction Coupling and Cardiac Contractile Force, Norwell: Kluwer Academic Publishers, 2002). The constructs according to the present invention, therefore, exhibit a decreased sensitivity to calcium versus normal cardiac tissue. However, once a threshold was obtained, the constructs exhibited similar increases in force production per unit rise in calcium as normal cardiac tissue. The inotropic response to external calcium is an indicator of the presence of functional calcium handling machinery.

Turning now to FIG. 7, the response to epinephrine was studied in four constructs according to the present invention by adding known concentrations of epinephrine to the CM, with the constructs still attached to the force transducer. By the start of this portion of testing, the constructs had ceased to exhibit spontaneous contractions. However, upon exchanging the fluid in the bath with fresh CM and with the addition of epinephrine to the culture bath to a concentration of 0.2 μg/ml (8 μl of a 0.1 mg/ml epinephrine solution in 4 ml of DMEM), the constructs re-initiated spontaneous contractions. The concentration of epinephrine was calculated based on the typical dosage given to an adult for cardiac resuscitation. The spontaneous contractility of the constructs was recorded for a period of 5 minutes after the addition of epinephrine. At some point (less than 60 seconds) following the onset of spontaneous contractile activity, the contraction of the constructs became quite rapid (resembling tachyarrhythmia). The constructs exhibited this behavior for approximately 60-90 seconds, ultimately exiting this rapid contraction pattern and re-entering a slower contraction behavior. When the constructs exited the rapid contraction pattern, a decrease in the force production along with the decreased frequency of contraction was noted. Thus, the constructs according to the present invention exhibited a chronotropic response to epinephrine, as one would expect in tissues of cardiac origin.

Cardiac tissue constructs according to the present invention were examined histologically. Constructs were fixed in 4% paraformaldehyde for 4 hours and stored in 70% ethanol. The constructs were prepared in an automated tissue processor (Shandon Hypercenter XP, Thermo Electron, Waltham, Mass.) and then paraffin embedded. Seven micron sections were cut and placed on Probeon Plus slides (Fisher Scientific Company, Pittsburgh, Pa.). Hematoxylin and eosin (H&E) staining was used for morphologic analysis of the constructs. To identify and localize endothelial cells, immunohistochemical staining for von Willebrand factor (vWF) was performed. The slides were heated in an oven at 60° C. for 20 minutes. The heated slides were then placed in 3 changes of xylene, 100% ethanol, 95% ethanol and buffer to prepare for staining. Antigen retrieval was performed for the slides designated for vWF staining by applying Proteinase K (DAKO Cytomation, Carpinteria Calif.) for 5 minutes at room temperature. The primary antibody (vWF antibody, polyclonal rabbit anti human (DAKO Cytomation) was incubated at a dilution of 1:250 at room temperature for 30 minutes. The Envision+Peroxidase Kit (DAKO Cytomation) was used for detection. Sections were viewed and photographed using a Nikon Axiophot inverted phase contrast microscope.

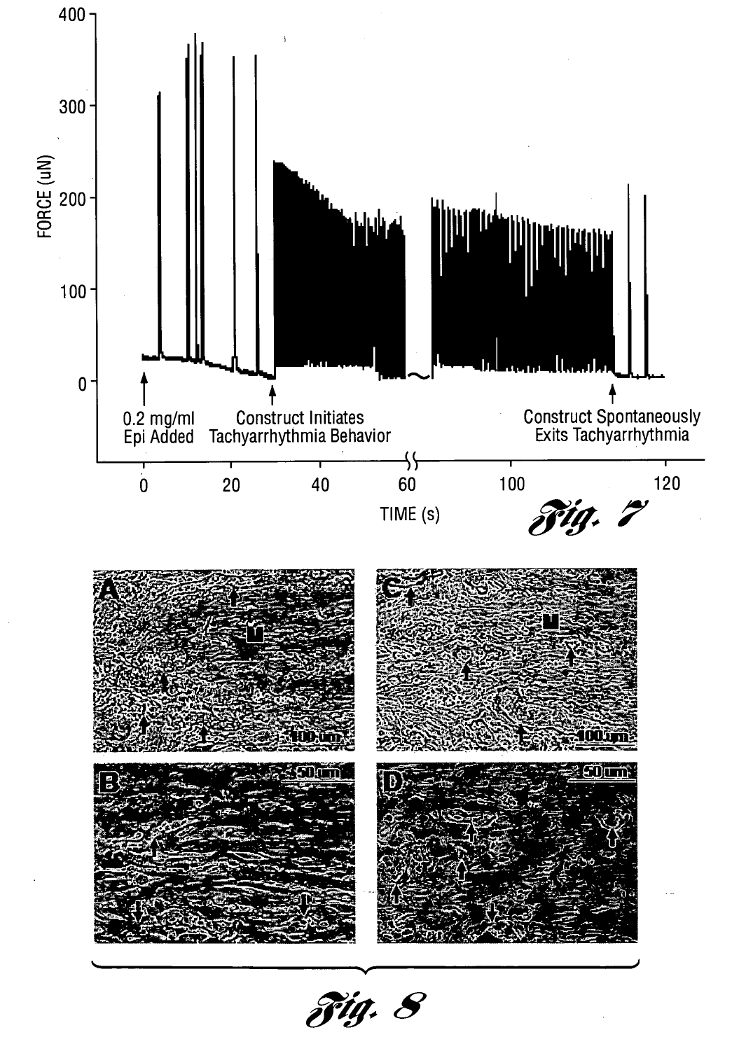

As depicted in FIGS. 8A and 8B, H&E staining showed large areas of easily recognizable muscle tissue permeated by multiple vascular channels of all sizes. Matrix deposition in the form of collagen was also present, which may have been laid down by fibroblasts introduced with the cardiac myocytes. Immunohistochemical staining for vWF highlighted the endothelium of this rich vascular network, which was filled with red blood cells (FIGS. 8C and D).

Cardiac constructs according to the present invention were also examined via electron microscopy (see Dennis and Kosnik, In Vitro Cellular & Developmental Biology, Animal 36(5): 327-335, 2000; Dennis et al., American Journal of Physiology—Cell Physiology 280(2): C288-C295, 2001). Constructs were fixed for 4 hours at 4° C. in Karnovsky's solution consisting of 0.1 M sodium cacodylate buffer with 3% formaldehyde and 3% glutaraldehyde (Electron Microscopy Sciences, Fort Washington, Pa.) at pH 7.4. Constructs were rinsed 3 times (30 min, 30 min and 4 hours) with cacodylate buffer, pH 7.4 with 7.5% sucrose. They were postfixed in 1% osmium tetroxide for 2 hours at room temperature, dehydrated in graded concentrations of ethanol and propylene oxide and embedded in EPON, Eponate 12 resin (Ted Pella Inc., Redding, Calif.). Embedded specimens were sectioned at 600 nm thick with a diamond knife on a Sorvall MT-5000 ultramicrotome (RMC, Inc., Tuscon, Ariz.). Ribbons of sections were mounted on copper grids, stained with uranyl acetate and lead citrate and examined using a Philips CM-100 transmission electron microscope. Photographs were obtained using a Kodak 1.6 Megaplus high resolution digital camera.

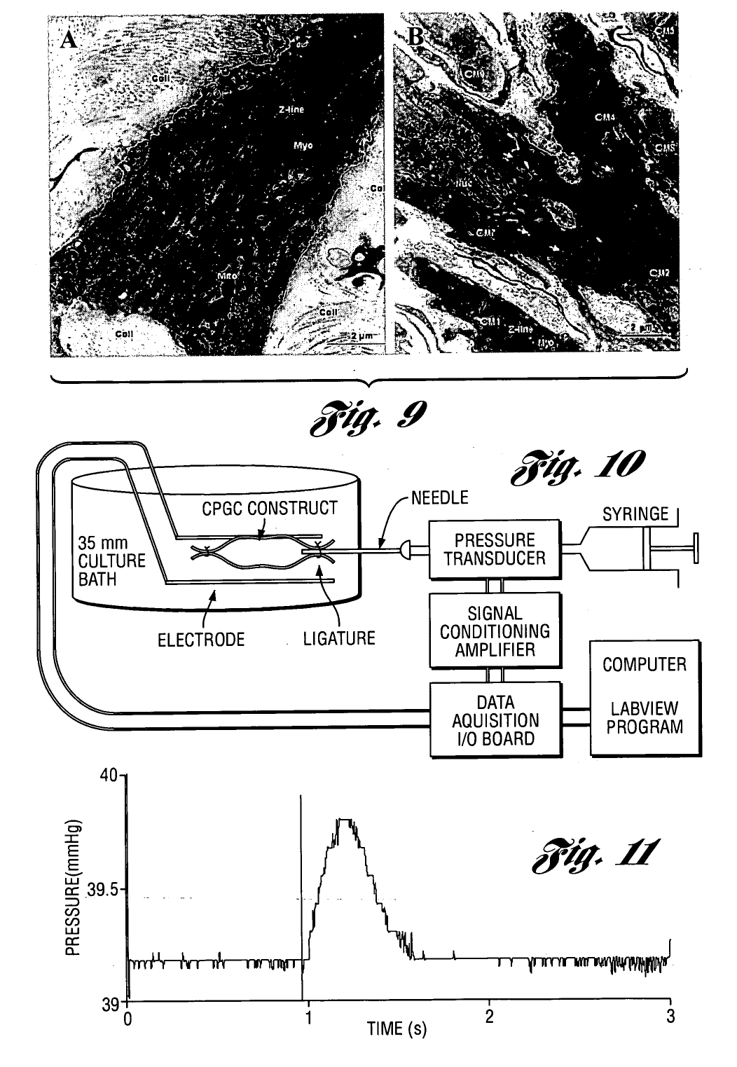

Electron micrographs of the constructs showed the presence of well-organized contractile proteins and equally spaced z-lines (FIG. 9A). The presence of intercalated discs was also confirmed (FIG. 9B). These structures and the high degree of intercellular connectivity give specific evidence of cardiac muscle machinery and organization.

As described above, the cardiac tissue constructs according to the present invention may act as a solid tissue patch. Additionally, the constructs may also be formed as tubular, flow-through constructs capable of generating intra-luminal pressure.

With reference to FIG. 10, a pressure monitoring system was assembled to measure the pressure changes within the lumen of the cardiac tissue constructs. The system includes a pressure transducer (such as MLT0670 Disposable BD Transducer, PowerLab, ADInstruments Inc, Colorado Springs, Colo.), signal conditioning amplifier, and data acquisition input/output (I/O) board (such as PCI-6040E, National Instruments, Austin, Tex.). The pressure transducer has a working range of approximately −50 to 300 mm Hg, and the response of the pressure transducer is approximately 5 μV/mm Hg per volt of excitation. The data I/O board was controlled by a custom made LabView (National Instruments) software program running on a personal computer. At the beginning of each pressure testing session, the pressure measurement levels were calibrated using a column of distilled water at heights of 1.0 and 0.1 meters at room temperature.

To test pressure for a cardiac tissue construct according to the present invention, one end of the construct was cannulated over a blunt-ended 27 gauge needle and ligated, such that the lumen of the construct was continuous with the lumen of the needle (FIG. 10). The needle was directly attached to the pressure transducer. The construct remained immersed in DMEM solution held at 37° C. during the testing session. A syringe was used to inject DMEM solution into the lumen of the pressure transducer, blunt-ended needle, and construct to ensure a continuous, internal volume of solution.

Excess fluid was allowed to drain from the unligated distal end (the left end as shown in FIG. 10) of the construct. Immediately following, the open distal end of the construct was ligated closed to prevent further passage of fluid. From this point on, the volume of the fluid in the lumen of the construct, needle and pressure transducer was constant. Any contractions of muscle cells in the walls of the cardiac tissue construct that applied pressure to the volume of solution within the lumen was recorded as an increase in pressure by the pressure transducer.

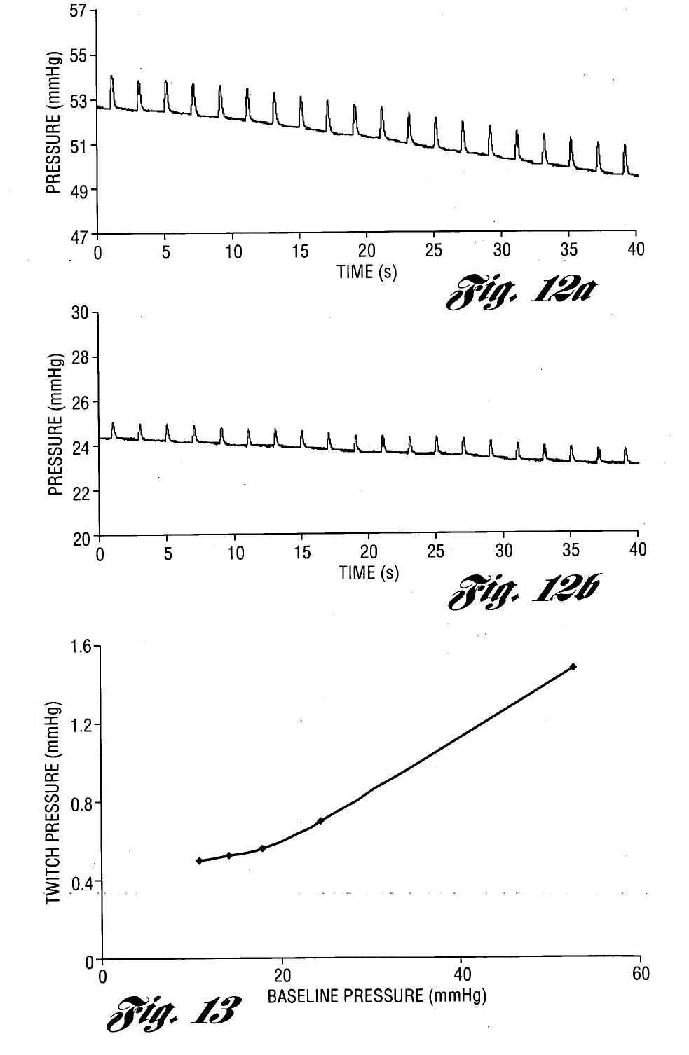

The custom made LabView program controlled the data I/O board to generate analog output voltage signals to electrically stimulate the construct. These output signals were amplified and delivered to the construct through stainless steel electrode wires lying parallel to the construct in the bath, one electrode on either side of the construct (FIG. 10). At the beginning of the pressure testing session, the spontaneous contractions of the construct were recorded in the absence of any electrical stimulation. This was followed by field stimulation of the construct to evaluate twitch pressure. The following stimulation pulse parameters were utilized: a single bipolar, balanced, square-wave pulse of ±7 volt amplitude and 1.2 ms duration. The pressure generated by the constructs in response to the electrical stimulation was measured by the pressure transducer and recorded in real time on the computer. Turning now to FIG. 11, the average twitch pressure obtained was 1.19±0.45 mm Hg (n=6), with a range of 0.75-2.0 mm Hg for the six constructs tested where each of the constructs succeeded in generating intra-luminal pressure. Therefore, the cardiac tissue constructs according to the present invention were able to generate intra-luminal pressure (radial force) upon electrical stimulation.

The starling behavior of the cardiac tissue constructs according to the present invention was also evaluated. As the baseline pressure decreased, a decrease in the twitch pressure of the constructs was observed, where an average decrease in baseline pressure of 56% (n=3) resulted in an average decrease in twitch pressure of 30% (n=3). For example, with reference to FIG. 12A, for one particular construct the baseline pressure started at 52.3 mm Hg while the twitch pressure was 1.473 mm Hg. As the baseline pressure decreased to 24.381 mm Hg, the twitch pressure decreased to 0.695 mm Hg (FIG. 12B). Therefore, Starling behavior was exhibited by the constructs of the present invention, in that increasing or decreasing the baseline pressure resulted in a similar change in the twitch pressure.

FIG. 13 depicts a starling curve for the cardiac tissue constructs according to the present invention, wherein the lumen of the construct was filled with liquid which was gradually allowed to escape into the testing bath. As the volume of liquid decreased, the baseline pressure as well as the active twitch pressure of the construct was evaluated. At the start of the testing, the construct had a baseline pressure of 52.633 mm Hg and a twitch pressure of 1.473 mm Hg. Towards the end of the testing, the baseline pressure had decreased to a value of 10.807 mm Hg and the twitch pressure had decreased to 0.497 mm Hg. The ability to modulate force production based on the preload is an important factor in the physiological regulation of the heart, and observation of this quality in the cardiac tissue constructs of the present invention is further indication of the functional organization of cardiac cells therein.

The method and constructs according to the present invention demonstrate the in vivo survival, vascularization, organization, and functionality of transplanted myocardial cells. The constructs resemble normal myocardial tissue in terms of their contractile properties, responsiveness to epinephrine and ionic calcium, length-force relationship, and their morphological characteristics. The method utilized herein to engineer cardiac tissue allows for the incorporation of a vascular network throughout the construct, promoting the survival of cells within the construct and allowing for an improvement in active force generation compared with avascular constructs. As engineering of cardiac replacement tissue proceeds, vascularization may be an increasingly important component in the development of three-dimensional structures.

These cardiac tissue constructs may be microsurgically transferable, as anastomosis of the vessels of the pedicle to a distant site or to another animal would be easily performed. The method according to the present invention also allows use of any of a number of different vascular sites. By increasing the chamber volume beyond that tested herein, the ratio of contractile muscle mass to vascular pedicle mass may be increased, thus possibly increasing force production.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A system for forming a cardiac tissue construct, comprising:
a chamber; and
cardiac myocytes provided within the chamber,
wherein the chamber is arranged to at least partially surround an intact blood vessel in vivo to facilitate formation of the three-dimensional cardiac tissue construct within the chamber.

2. The system according to claim 1, wherein the cardiac myocytes are provided within a suspension including a fibrin gel.

3. The system according to claim 2, wherein the suspension further includes thrombin.

4. The system according to claim 3, wherein the fibrin gel includes a ratio of approximately 200 μl of 3.5 mg/ml fibrinogen to approximately 5 μl of 200 U/ml of thrombin.

5. The system according to claim 2, wherein the suspension has a concentration of approximately 20 million cells to 50 μl of fibrin gel, wherein the cells include cardiac myocytes.

6. The system according to claim 1, wherein the chamber includes a tube.

7. The system according to claim 6, wherein the tube is constructed from silicone.

8. The system according to claim 1, wherein the chamber includes an opening therein to aid in placement of the chamber with respect to the blood vessel.

9. A method of forming a cardiac tissue construct, comprising:
providing a chamber;
placing cardiac myocytes within the chamber, the chamber arranged to at least partially surround an intact blood vessel in vivo to allow formation of the three-dimensional cardiac tissue construct within the chamber.

10. The method according to claim 9, wherein placing cardiac myocytes includes providing the cardiac myocytes within a suspension including a fibrin gel.

11. The method according to claim 10, wherein the suspension further includes thrombin.

12. The method according to claim 9, wherein the fibrin gel includes a ratio of approximately 200 μl of 3.5 mg/ml fibrinogen to approximately 5 μl of 200 U/ml of thrombin.

13. The method according to claim 9, wherein the suspension has a concentration of approximately 20 million cardiac myocytes to 50 μL of fibrin gel.

14. The method according to claim 9, further comprising implanting the chamber in a host such that the chamber includes the cardiac myocytes and the blood vessel.

15. The method according to claim 14, wherein the blood vessel includes an artery.

16. The method according to claim 14, wherein implanting the chamber includes separating the chamber along an opening provided therein and placing the blood vessel at least partially inside the chamber.

17. The method according to claim 9, further comprising incubating the chamber and contained cardiac myocytes in vivo for a period of time.

18. The method according to claim 17, further comprising removing the construct from the chamber following incubation.

19. The method according to claim 9, wherein providing the chamber includes providing a silicone tube.

20. The method according to claim 9, further comprising forming the cardiac tissue construct into a tube having a lumen.

21. A cardiac tissue construct, comprising:
cardiac myocytes provided within a chamber which is incubated in vivo at least partially surrounding an intact blood vessel in order to form the three-dimensional cardiac tissue construct.

22. The construct according to claim 21, wherein the cardiac myocytes are provided within a suspension including a fibrin gel.

23. The construct according to claim 22, wherein the suspension further includes thrombin.

24. The construct according to claim 22, wherein the fibrin gel includes a ratio of approximately 200 μl of 3.5 mg/ml fibrinogen to approximately 5 μl of 200 U/ml of thrombin.

25. The construct according to claim 22, wherein the suspension has a concentration of approximately 20 million cells to 50 μl of fibrin gel, wherein the cells include cardiac myocytes.

26. The construct according to claim 21, wherein the chamber includes generally cylindrical tubing.

27. The construct according to claim 21, wherein the chamber includes an opening therein to aid in placement of the chamber with respect to the blood vessel.

28. The construct according to claim 21, wherein the construct is electrically excitable.

29. The construct according to claim 21, wherein the construct is capable of generating force upon stimulation.

30. The construct according to claim 21, wherein when formed into a tube having a lumen, the construct is capable of generating intra-luminal pressure upon stimulation.

31. The construct according to claim 30, wherein a change in a baseline pressure of the construct results in a similar change in an active twitch pressure of the construct.

32. The construct according to claim 21, wherein a cross-section of the construct includes muscle tissue permeated by vascular channels.

33. The cardiac tissue construct according to claim 19, wherein the construct exhibits an inotropic response to external calcium and a chronotropic response to epinephrine.
